(19) United States
(12) Patent Application Publication
(10) Pub. No.: US 2023/0272456 A1
(43) Pub. Date: Aug. 31, 2023

(54) CASCADING AMPLIFICATION FOR CHEMICAL AND BIOSENSING

(71) Applicant: SiPhox, Inc., Cambridge, CA (US)

(72) Inventor: Michael Dubrovsky, Cambridge, MA (US)

(21) Appl. No.: 18/008,130

(22) PCT Filed: Jun. 3, 2021
(86) PCT No.: PCT/US2021/035710

§ 371 Date: Dec. 2, 2022

Related U.S. Application Data
(60) Provisional application No. 63/043,224, filed on Jun. 24, 2020.
(60) Provisional application No. 63/034,205, filed on Jun. 3, 2020.

(51) Int. Cl. C12Q 1/6816; G01N 33/573; G01N 21/552; G01N 33/563

(52) U.S. Cl. CPC C12Q 1/6816; G01N 33/573; G01N 21/553; G01N 33/563; G01N 2021/258

(57) ABSTRACT

The invention relates generally to systems and methods for assaying a biological sample suspected of comprising a target biomolecule using cascading amplification.

### CROSS-REFERENCE TO RELATED APPLICATIONS

This application claims priority to and the benefit of U.S. Provisional Application No. 63/043,224 filed 24 Jun. 2020, and U.S. Provisional Application No. 63/034,205 filed 3 Jun. 2020, each of which is incorporated herein by reference in its entirety.

### FIELD OF THE INVENTION

The following disclosure is directed to methods and systems for cascading amplification for chemical and biosensing.

### BACKGROUND

Many sensing approaches can detect or bind small quantities of analyte but suffer from the inability to read out the resulting signal. On the other hand, sensors with very sensitive readout tend to be non-specific, leading to false positives.

In recent years, sensitive, reliable, and facile methods for the detection of biological material (e.g., RNA/DNA, antibodies, antigens, etc.) has grown in importance for medical diagnosis and a range of other biological fields. Techniques for biosensing use a range of thermal, optical, electrical, electrochemical, and or gravimetric methods. Various amplification strategies have been developed for biosensing that may permit sensing of biological material present in small concentrations. For example, polymerase chain reaction (PCR) represents the most commonly used method for amplification of nucleic acid detection but is limited by the need for expensive equipment and skilled technicians. New systems and methods for sensitive, specific, and reliable detection of biological materials are needed.

### SUMMARY OF THE INVENTION

Generally, the present disclosure discusses methods and designs for improving sensing systems via cascading chemical, biological, biochemical, electrical, optical, magnetic and other cascading amplification steps.

In some embodiments, described herein are methods for assaying a biological sample suspected of comprising a target biomolecule, the method comprising the steps of contacting the sample with a primary sensing agent, wherein the primary sensing agent is activated in the presence of said target biomolecule; providing conditions for activating a secondary sensing agent in the presence of said activated primary sensing agent, wherein said activated primary sensing agent comprises a first catalytic activity that results in activation of said secondary sensing agent comprising a second catalytic activity; providing conditions for detecting a signal agent, wherein said activated secondary sensing agent comprises a second catalytic activity for a reaction that results in detection of said signal agent; and detecting a quantity of a signal generated by said signal agent, thereby assaying said biological sample for said target biomolecule. In some embodiments, the activation of said secondary sensing agent comprises cleaving a linker binding said secondary sensing agent to a surface. In some embodiments, the primary sensing agent and/or said secondary second sensing agent is an enzyme. In some embodiments, the primary sensing agent and/or said secondary sensing agent is a cleavage enzyme. In some embodiments the target biomolecule is a biological marker. In some embodiments, the target biomolecule is a dsDNA, a ssDNA, a RNA, a peptide, a protein, an antigen, or a pathogen. In some embodiments, the target biomolecule comprises a RNA or a DNA. In some embodiments, the biological sample is blood, cord blood, saliva, tissue, mucus, nasal swab, or urine.

In some embodiments, disclosed herein are methods for detecting a target biomolecule in a biological sample, the method comprising contacting the sample with a primary sensing agent, wherein the primary sensing agent is activated by said target biomolecule; contacting said activated primary sensing agent with a secondary sensing agent, wherein said activated primary sensing agent catalyzes a first reaction that results in activation of said secondary sensing agent comprising a second catalytic activity; contacting said activated secondary sensing agent with a signal agent, wherein said activated secondary sensing agent comprises a second catalytic activity for a second reaction that results in detection of said signal agent; and detecting a signal generated by said signal agent, thereby detecting said target biomolecule in said biological sample. In some embodiments, the target biomolecule is a biological marker. In some embodiments the target biomolecule is a dsDNA, a ssDNA, a RNA, a peptide, a protein, an antigen, or a pathogen. In some embodiments the target biomolecule comprises a RNA or a DNA. In some embodiments, the biological sample is blood, cord blood, saliva, tissue, mucus, nasal swab, or urine. In some embodiments, the primary sensing agent comprises a CRISPR enzyme. In some embodiments, the CRISPR enzyme comprises a Cas12 complex or a Cas13 complex. In some embodiments, the CRISPR enzyme comprises a Cas12a complex. In some embodiments, the CRISPR enzyme comprises a Cas13 selected from the group consisting of a LwaCas13a, a CcaCas13b. a LbaCas13a, and a PsmCas13b. In some embodiments, the secondary sensing agent is an enzyme selected from the group consisting of a restriction enzyme, a hydrolase, a peroxidase, a lyase, a ligase, a glutathione S-transferase, and a SpyTag/SpyCatcher. In some embodiments, the secondary sensing agent has nuclease activity. In some embodiments, the secondary sensing agent comprises a CRISPR enzyme. In some embodiments the CRISPR enzyme comprises a Cas12 complex or a Cas13 complex. In some embodiments, the CRISPR enzyme comprises a Cas12a complex. In some embodiments, wherein the CRISPR enzyme comprises a Cas13 selected from the group consisting of a LwaCas13a, a CcaCas13b. a LbaCas13a, and a PsmCas13b. In some embodiments, the secondary sensing agent is an binding protein selected from the group consisting of an avidin, a maltose-binding protein, and a chitin-binding protein. In some embodiments, the activation of said secondary sensing agent comprises cleaving a first linker binding said secondary sensing agent to a first surface. In some embodiments, the activation of said secondary sensing agent comprises cleaving an inactivating linker on said secondary sensing agent. In some embodiments, the linker comprises ssDNA, dsDNA, RNA, or a combination thereof. In some embodiments, the linker comprises an oligonucleotide or a modified oligonucleotide. In some embodiments, the linker comprises a cleavage or recognition site for the primary sensing agent. In some embodiments, the recognition or cleavage site comprises a TTA sequence or a TTA sequence repeat. In some embodiments, the recognition or cleavage site comprises a sequence selected from the group consisting of AU, UC, AC, and GA. In some embodiments, the linker is a peptide linker. In some embodiments, the secondary sensing agent comprises a cleavable oligonucleotide linker. In some embodiments, the secondary sensing agent is attached to a surface support via the linker. In some embodiments, the surface support is located on a paper flow strip, a bead, or a microfluidic channel. In some embodiments, the method further comprises detaching the secondary sensing agent from the surface support by cleaving the cleavable oligonucleotide linker with the activated primary sensing agent. In some embodiments, the oligonucleotide linker further comprises a scrambled sequence. In some embodiments, the scrambled sequence remains attached to the secondary sensing agent after linker cleavage by a unrelated enzyme in the biological sample. In some embodiments, the secondary sensing agent comprising the scrambled sequence is filtered out of the sample and not detected. In some embodiments, the filtering comprises hybridizing the recognition sequence with a complementary or partially complementary oligonucleotide attached to a surface. In some embodiments, the detecting step is performed by a detector. In some embodiments, the detector comprises a photonic or plasmonic waveguide. In some embodiments, the waveguide is an optical ring resonator or a unbalanced Mach-Zehnder interferometer. In some embodiments, the second reaction results in a reaction product and the reaction product is captured by components bound to a detector, thereby resulting in a measurable signal. In some embodiments, the detecting step comprises detecting changes in refractive index, electrical voltage, electrical current, optical absorbance, color, fluorescence, weight, melting temperature, or chemiluminescence. In some embodiments, the detecting step comprises detecting changes in refractive index. In some embodiments, the changes in refractive index are at the surface of a photonic or plasmonic waveguide. In some embodiments, the method further comprises contacting a binding agent or probe located on the detector with the reaction product. In some embodiments, the probe or binding agent is an antibody, an antigen, or an aptamer. In some embodiments the probe or binding agent comprises an optically active component. In some embodiments, the optically active component is a plasmonic nanoparticle, a gold nanoparticle, a quantum dot, or a fluorophore. In some embodiments, the component of said target biomolecule activates a cleaving component in the primary sensing agent. In some embodiments, the secondary sensing agent is dried and is located within a high-surface area. In some embodiments, the biological sample is placed in a reaction chamber comprising a high surface area and volume, and comprising a plurality of primary sensing agents. In some embodiments, the plurality of primary sensing agents in the reaction chamber is activated by the target biomolecule. In some embodiments, the method further comprises, moving the sample through a microfluidic channel for sample detection. In some embodiments, the method further comprises a filter for filtering the sample before sample detection. In some embodiments, the method further comprises a tertiary sensing agent bound by a second linker to a second surface, wherein said activated primary sensing agent catalyzes a third reaction that results in activation of said tertiary sensing agent comprising a third catalytic activity. In some embodiments, the activated secondary sensing agent binds and cleaves the second linker and the activated tertiary sensing agent binds and cleaves the first linker, thereby cleaving a plurality of secondary and tertiary sensing agents from the first and second surfaces.

In some embodiments, disclosed herein are systems for detecting a target biomolecule in a biological sample, the system comprising: a primary sensing agent activatable by the target biomolecule; a secondary sensing agent, comprising a linker comprising a cleavage site cleavable by the activated primary sensing agent; and wherein the secondary sensing agent is activatable by the linker cleavage; a detector, wherein the detector is capable of sensing the activated secondary sensing agent, thereby detecting the presence of said target biomolecule in said biological sample. In some embodiments. In some embodiments, the target biomolecule is a biological marker. In some embodiments, the target biomolecule is selected from the group consisting of a dsDNA, a ssDNA, a RNA, a peptide, a protein, an antigen, and a pathogen. In some embodiments, the target biomolecule comprises a RNA or a DNA. In some embodiments, the biological sample is selected from the group of blood, cord blood, saliva, mucus, tissue, nasal swab, and urine. In some embodiments, the primary sensing agent comprises a CRISPR enzyme. In some embodiments, the CRISPR enzyme comprises a Cas12 complex or a Cas13 complex. In some embodiments, the CRISPR enzyme comprises a Cas12a complex. In some embodiments, the CRISPR enzyme comprises a Cas13 selected from the group consisting of a LwaCas13a, a CcaCas13b. a LbaCas13a, and a PsmCas13b. In some embodiments, the secondary sensing agent is an enzyme selected from the group consisting of a restriction enzyme, a hydrolase, a peroxidase, a lyase, a ligase, a glutathione S-transferase, and a SpyTag/SpyCatcher. In some embodiments, the secondary sensing agent has nuclease activity. In some embodiments, the secondary sensing agent comprises a CRISPR enzyme. In some embodiments, the CRISPR enzyme comprises a Cas12 complex or a Cas13 complex. In some embodiments, the CRISPR enzyme comprises a Cas12a complex. In some embodiments, the CRISPR enzyme comprises a Cas13 selected from the group consisting of a LwaCas13a, a CcaCas13b. a LbaCas13a, and a PsmCas13b. In some embodiments, the secondary sensing agent is an binding protein selected from the group consisting of an avidin, a maltose-binding protein, and a chitin-binding protein. In some embodiments, the activation of said secondary sensing agent comprises cleaving a first linker binding said secondary sensing agent to a first surface. In some embodiments, the activation of said secondary sensing agent comprises cleaving an inactivating linker on said secondary sensing agent. In some embodiments, the linker comprises ssDNA, dsDNA, RNA, or a combination thereof. In some embodiments, the linker comprises an oligonucleotide or a modified oligonucleotide. In some embodiments, the linker comprises a cleavage or recognition site for the primary sensing agent. In some embodiments, the recognition or cleavage site comprises a TTA sequence or a TTA sequence repeat. In some embodiments, the recognition or cleavage site comprises a sequence selected from the group consisting of AU, UC, AC, and GA. In some embodiments, the linker is a peptide linker. In some embodiments, the secondary sensing agent comprises a cleavable oligonucleotide linker. In some embodiments, the secondary sensing agent is attached to a surface support via the linker. In some embodiments, the surface support is located on a paper flow strip, a bead, or a microfluidic channel. In some embodiments, the system further comprises, detaching the secondary sensing agent from the surface support by cleaving the cleavable oligonucleotide linker with the activated primary sensing agent. In some embodiments, the oligonucleotide linker further comprises a scrambled sequence. In some embodiments, the scrambled sequence remains attached to the secondary sensing agent after linker cleavage by a unrelated enzyme in the biological sample. In some embodiments, the secondary sensing agent comprising the scrambled sequence is filtered out of the sample and not detected. In some embodiments, the filtering comprises hybridizing the recognition sequence with a complementary or partially complementary oligonucleotide attached to a surface. In some embodiments, the detector comprises a photonic or plasmonic waveguide. In some embodiments, the waveguide is an optical ring resonator or a unbalanced Mach-Zehnder interferometer. In some embodiments, the detector measures changes in refractive index, electrical voltage, electrical current, optical absorbance, color, fluorescence, weight, melting temperature, or chemiluminescence. In some embodiments, the detector measures changes in refractive index. In some embodiments, the changes in refractive index are at the surface of a photonic or plasmonic waveguide. In some embodiments, the system further comprises a binding agent or probe located on the detector. In some embodiments, the probe or binding agent is an antibody, an antigen, or an aptamer. In some embodiments, the probe or binding agent comprises an optically active component. In some embodiments, the optically active component is a plasmonic nanoparticle, a gold nanoparticle, a quantum dot, or a fluorophore. In some embodiments, the component of said target biomolecule is capable of activating a cleaving component in the primary sensing agent. In some embodiments, the secondary sensing agent is dried and is located within a high-surface area. In some embodiments, the system comprises a reaction chamber comprising a high surface area and volume, and comprising a plurality of primary sensing agent for placing the biological sample. In some embodiments, the plurality of primary sensing agents in the reaction chamber is activatable by the target biomolecule. In some embodiments, the system further comprises, a microfluidic channel for moving the sample for sample detection. In some embodiments, the system further comprises a filter, for filtering the sample before sample detection. In some embodiments, the system further comprises a tertiary sensing agent bound by a second linker to a second surface, wherein said activated primary sensing agent is capable of catalyzing a third reaction resulting in activation of said tertiary sensing agent comprising a third catalytic activity. In some embodiments, the activated secondary sensing agent is capable of binding and cleaving the second linker and the activated tertiary sensing agent is capable of binding and cleaving the first linker, thereby resulting in cleavage of a plurality of secondary and tertiary sensing agents from the first and second surfaces.

In some embodiments, disclosed herein are methods for assaying a biological sample suspected of comprising a target biomolecule, comprising: obtaining a biological sample in a cartridge, wherein the cartridge comprises a sensor photonic integrated subcircuit; contacting the sample with a primary sensing agent, wherein the primary sensing agent is activated in the presence of said target biomolecule; providing conditions for activating a secondary sensing agent in the presence of said activated primary sensing agent, wherein said activated primary sensing agent comprises a first catalytic activity that results in activation of said secondary sensing agent comprising a second catalytic activity; providing conditions for detecting a signal agent, wherein said activated secondary sensing agent comprises a second catalytic activity for a reaction that results in detection of said signal agent; positioning the cartridge relative to an interrogator photonic circuit such that the cartridge is optically coupled with the interrogator photonic circuit, wherein the interrogator photonic circuit comprises (i) a light source configured to generate light, (ii) a waveguide configured to carry the light, and (iii) a photodetector configured to detect said light after passing through said waveguides; and determining, via the light, a characteristic of the biological sample in the cartridge thereby detecting a quantity of a signal generated by said signal agent, thereby assaying said biological sample for said target biomolecule. In some embodiments, the characteristic of the biological sample is determined based on a change in resonance, interference, or absorption caused by the biological sample.

### DESCRIPTION OF THE DRAWINGS

The invention can be more completely understood with reference to the following drawings.

FIG. 1 illustrates an example of cascading amplification where a sensing target (analyte) activates a primary sensing agent which goes on to activate a plurality of secondary or tertiary sensing agents via a cascade.

FIG. 2 illustrates an example of cascading amplification where the sensing target activates the primary sensing agent (Step I), the activated primary sensing agent cleaves secondary sensing agents from a surface support (Step II), the liberated secondary sensing agent cleaves tertiary sensing agents from the detector (Step III, resulting in a measurable signal via cleavage of probes from a detector. The sensing target and the primary sensing agent are present in solution.

FIG. 3 illustrates an example of cascading amplification where the activated sensing agent cleaves surface affixed binding agents from a surface, and the cleaved binding agent binds to a detector, resulting in a measurable signal.

FIG. 4 illustrates an example of cascading amplification where the activated sensing agent cleaves surface affixed binding agents from a surface, and the cleaved binding agent is captured by components bound to the detector, resulting in a measurable signal.

FIG. 5 illustrates an example of a fluid flow scenario where dried secondary sensing agents are immobilized within a high-surface area material, are cleaved from then surface by the activated sensing agent and flow towards dried tertiary sensing agents immobilized within a high-surface area material. The interaction between the secondary and tertiary sensing agents results in a detectable signal (e.g., colorimetric).

FIG. 6 illustrates an example of a surface that can be functionalized with probes or agents then capped to prevent unwanted attachment of the agent, probe, or sensing target to the surface.

FIG. 7 illustrates an example where a specific cleavage of a particular ssDNA or RNA oligo sequence barcode (e.g., identity) may help distinguish the presence of activated/released agents that were released/activated via cleavage at the correct sequence from other agents within the system (e.g., inadvertent cleavage products from side reactions or random cleavage) that have a different remaining tail of ssDNA/RNA after cleavage/activation.

FIG. 8 illustrates an example where additional amplification may be achieved by making the linkage that immobilizes or inactivates agent A cleavable/removable by agent B, and the linkage that immobilizes or inactivates agent B cleavable/removable by agent A. Both linkages or one of them may be cleavable or removable by the agent in the previous step of the cascade (e.g., the sensing agent or secondary/tertiary/etc. agent).

FIG. 9 illustrates an example for a sensing system wherein the sample is placed in a reaction chamber with high surface area and volume to facilitate amplification and interaction with the entire sample volume before moving the sample into a microfluidic channel for sample detection. A filter may be added to remove parts of the sample or amplification reagents.

FIG. 10 illustrates an example for a surface with immobilized secondary sensing agents, wherein the secondary sensing agent is attached to the surface with a cleavable linker. Additional amplification may be achieved by designing the linker such that it is cleavable/removable by the primary sensing agent as well as the secondary sensing agent in the previous step of the cascade.

FIG. 11 illustrates an example for a surface functionalized with agent A which may then capture and/or cleave agent B, resulting in a detectable signal by a detector.

FIG. 12 illustrates an example, wherein an analyte or activated sensing agent A interacts with a sensing agent B forming a complex C. and wherein complex C cleaves probes from a sensor or further sensing agents from a surface.

FIG. 13 illustrates an example, wherein a sensing agent may act as binders between 2 or more other molecules such that, once the agent is activated, it goes on to create an activated complex (e.g., an enzyme).

FIG. 14 illustrates an example, wherein an activated sensing agent (agent B) may cleave linkages that connect agents (agents A and/or C which may feature another complex/molecule/particle). Cleavage of this group of connected agents may result in activated agents (activated agent A for example).

### DETAILED DESCRIPTION

Disclosed herein, inter alia, are methods for assaying biological samples comprising target molecules using cascading amplification, comprising the steps of contacting a sample with a primary sensing agent, wherein the primary sensing agent is activated in the presence of the target biomolecule; providing conditions for activating a secondary sensing agent in the presence of said activated primary sensing agent, wherein the activated primary sensing agent comprises a first catalytic activity that results in activation of the secondary sensing agent comprising a second catalytic activity; providing conditions for detecting a signal agent, wherein the activated secondary sensing agent comprises a second catalytic activity for a reaction that results in detection of the signal agent; and detecting a quantity of a signal generated by the signal agent, thereby assaying the biological sample for said target biomolecule.

Disclosed herein, inter alia, are methods for assaying a biological sample suspected of comprising a target biomolecule, comprising the steps of: obtaining a biological sample in a cartridge, wherein the cartridge comprises a sensor photonic integrated subcircuit; contacting the sample with a primary sensing agent, wherein the primary sensing agent is activated in the presence of said target biomolecule; providing conditions for activating a secondary sensing agent in the presence of said activated primary sensing agent, wherein said activated primary sensing agent comprises a first catalytic activity that results in activation of said secondary sensing agent comprising a second catalytic activity; providing conditions for detecting a signal agent, wherein said activated secondary sensing agent comprises a second catalytic activity for a reaction that results in detection of said signal agent; positioning the cartridge relative to an interrogator photonic circuit such that the cartridge is optically coupled with the interrogator photonic circuit, wherein the interrogator photonic circuit comprises (i) a light source configured to generate light, (ii) a waveguide configured to carry the light, and (iii) a photodetector configured to detect said light after passing through said waveguides; and determining, via the light, a characteristic of the biological sample in the cartridge thereby detecting a quantity of a signal generated by said signal agent, thereby assaying said biological sample for said target biomolecule.

Disclosed herein, inter alia, are systems for detecting a target biomolecule in a biological sample, the system comprising: a primary sensing agent activatable by the target biomolecule; a secondary sensing agent, comprising a linker comprising a cleavage site cleavable by the activated primary sensing agent; and wherein the secondary sensing agent is activatable by the linker cleavage; a detector, wherein the detector is capable of sensing the activated secondary sensing agent, thereby detecting the presence of said target biomolecule in said biological sample.

### Cascading Amplification Methods and Systems

As used herein, an “analyte” is understood to refer to an entity that can activate a sensing agent. Analytes comprise, but are not limited to a solution molecule, a complex or a biological cell in solution, or a gas. In some embodiments, an analyte comprises a target biomolecule. In some embodiments the analyte comprises a chemical or biochemical molecule. In some embodiments the analyte comprises DNA or RNA.

In some embodiments the analyte is a biological sample. In some embodiments the biological sample comprises, but is not limited to blood, cord blood, saliva, mucus, tissue, nasal swab, or urine.

As used herein, a “target biomolecule”, or “sensing target” are used interchangeably. A target biomolecule is a biomolecule present in a biological sample. A target biomolecule can comprise any biological markers. Target biomolecules comprise, but are not limited to dsDNA, ssDNA, RNA, peptides, proteins, antigens, or pathogens. In some embodiments, target biomolecules can be viral RNA, DNA, or antigen. In some embodiments the target biomolecule comprises an enzyme. In some embodiments the target biomolecule is DNA.

As used herein, “oligonucleotide,” “polynucleotide,” or “nucleic acid,” are used interchangeably herein, and refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by I1 P(O)S (“thioate”), P(S)S (“dithioate”). (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As used herein, the term “complementarity” refers to the ability of a nucleic acid sequence to form hydrogen bond(s) with another nucleic acid sequence (e.g, through traditional Watson-Crick base pairing, Hoogsteen hydrogen bonding or wobble hydrogen bonding). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

As used herein, “Clustered Regularly Interspaced Short Palindromic Repeats (CRISPR)” and “cas (CRISPR-associated)” genes comprise an adaptive immune system that provides acquired resistance against invading foreign nucleic acids in bacteria and archaea (Barrangou et al. (2007) Science 315:1709-12). CRISPR-Cas systems are classified into two distinct classes, Class 1 and Class 2 and are described in detail in Koonin et al., Curr Opin Microbiol. (2017) 37:67-78. Class 1 CRISPR-Cas systems comprise the multiprotein effector complexes (Type I (Cascade effector complex), III (Cmr/Csm effector complex), and IV), and Class 2 CRISPR-Cas systems comprise single effector proteins (Type II (Cas9), V (for example Cas12a, previously referred to as Cpf1), and VI (for example Cas13a, previously referred to as C2c2)). Additionally, CRISPR-Cas systems comprise one or more RNAs termed crRNAs or guide RNA (gRNA) that are derived from the CRISPRs. Some CRISPR-Cas systems comprise one or more RNAs termed tracrRNA derived from another locus. During an infection, CRISPR-Cas systems specifically incorporate foreign viral DNA or RNA sequences (spacers) into CRISPRs.

Type V CRISPR-Cas12 systems comprise subtypes Cas12a and Cas12b. Cas12a is also known as Cpf1 and Cas12b is known as C2c1. Typically the total guide length is 42-44 nucleotides. The typical spacer length is 18-25 nucleotides. Cas12 is a compact and efficient enzyme that creates staggered cuts in dsDNA. Cas12 processes its own guide RNAs, leading to increased multiplexing ability. Cas12 has also been engineered as a platform for epigenome editing, and it was recently discovered that Cas12a can indiscriminately chop up single-stranded DNA once activated by a target DNA molecule matching its spacer sequence.

Type VI CRISPR-Cas13 systems comprise subtypes Cas13a, Cas13b, Cas13c, and Cas13d). Cas13d is also known as C2c2 or CasRx (described in Abudavyeh et al.. Science (2016) 353:6299). Typically the total guide length is 52-66 nucleotides. The typical spacer length is 22-30 nucleotides. Cas13 is an outlier in the CRISPR enzymes because it targets RNA, not DNA. Once it is activated by a ssRNA sequence bearing complementarity to its crRNA spacer, it activates a nonspecific RNase activity and destroys all nearby RNA regardless of their sequence.

As used herein, a “sensing agent” is understood to be an entity that can be activated by an analyte or another activated sensing agent. Sensing agents comprise, but are not limited to enzymes or protein binding proteins. Exemplary sensing agents are listed in Table 1.

TABLE 1
Examples of sensing agents

| Type | Activity | Examples |
|---|---|---|
| Restriction | Recognize and | Nucleases, Types I-V, |
| Enzymes | cleave DNA | CRISPR |
| Hydrolases | Use water to cleave | esterases, proteases, |
| | chemical bonds | glycosidases, |
| | | nucleosidases, lipases |
| Oxidoreductases | Oxidize substrate by | dehydrogenase |
| | reducing electron | |
| | acceptor | |
| Peroxidases | Break up peroxides | catalase |
| Lyases | Use functional groups | decarboxylase |
| | to break double bonds | |
| Ligases | Joins two large | DNA Ligase |
| | molecules by forming | |
| | chemical bond | |
| Avidins | Biotin-binding protein | streptavidin |
| Glutahione | Conjugate glutathione | GSTA1 |
| STransferase | to electrophilic | |
| | compounds | |
| Maltose-binding | Binds to maltose | MBP |
| protein | or amylose | |
| Chitin-binding protein | Binds chitin | |
| SpyTag/SpyCatcher | Reaction forms | |
| | isopeptide bond | |
| | between pair | |

In some embodiments, the sensing agent has catalytic activity. Catalytic activity can be an enzymatic activity such as but not limited to, hydrolase, oxidoreductase, transferase, lyase, isomerase, and ligase activity.

In some embodiments, the sensing agent can be a nuclease. In some embodiments, the nuclease can be a ribonuclease or a deoxyribonuclease. In some embodiments, the nuclease can be a CRISPR enzyme. In some embodiments, the CRISPR enzyme can be a Type V or Type VI CRISPR enzyme. In some embodiments, the Type V or Type VI CRISPR enzyme is a Cas12 or a Cas13 enzyme. In some embodiments, the Cas13 enzyme is a LwaCas13a, CcaCas13b, LbaCas13a, or a PsmCas13b. In some embodiments the CRISPR enzyme is a Cas12a complex.

In some embodiments, the primary sensing agent can be a nuclease. In some embodiments, the nuclease can be a ribonuclease or a deoxyribonuclease. In some embodiments, the nuclease can be a CRISPR enzyme. In some embodiments, the CRISPR enzyme can be a Type V or Type VI CRISPR enzyme. In some embodiments, the Type V or Type VI CRISPR enzyme is a Cas12 or a Cas13 complex. In some embodiments, the Cas13 enzyme is a LwaCas13a, CcaCas13b, LbaCas13a, or a PsmCas13b. In some embodiments the CRISPR enzyme is a Cas12a enzyme.

In some embodiments, the secondary sensing agent is an enzyme, comprising, but not limited to a restriction enzyme, a hydrolase, a peroxidase, a lyase, a ligase, a glutathione S-transferase, and a SpyTag/SpyCatcher. In some embodiments the secondary sensing agent is a binding protein comprising, but not limited to an avidin, a maltose-binding protein, and a chitin-binding protein. In some embodiments, the secondary sensing agent can be a nuclease. In some embodiments, the nuclease can be a ribonuclease or a deoxyribonuclease. In some embodiments, the nuclease can be a CRISPR enzyme. In some embodiments, the CRISPR enzyme can be a Type V or Type VI CRISPR enzyme. In some embodiments, the Type V or Type VI CRISPR enzyme is a Cas12 or a Cas13 complex. In some embodiments, the Cas13 enzyme is a LwaCas13a, CcaCas13b, LbaCas13a, or a PsmCas13b. In some embodiments the CRISPR enzyme is a Cas12a enzyme.

In some embodiments, sensing agents comprise one or more primary, secondary, tertiary etc. sensing agents.

In some embodiments, the tertiary sensing agent is an enzyme, comprising, but not limited to a restriction enzyme, a hydrolase, a peroxidase, a lyase, a ligase, a glutathione S-transferase, and a SpyTag/SpyCatcher. In some embodiments the secondary sensing agent is a binding protein comprising, but not limited to an avidin, a maltose-binding protein, and a chitin-binding protein. In some embodiments, the secondary sensing agent can be a nuclease. In some embodiments, the nuclease can be a ribonuclease or a deoxyribonuclease. In some embodiments, the nuclease can be a CRISPR enzyme. In some embodiments, the CRISPR enzyme can be a Type V or Type VI CRISPR enzyme. In some embodiments, the Type V or Type VI CRISPR enzyme is a Cas12 or a Cas13 complex. In some embodiments, the Cas13 enzyme is a LwaCas13a, CcaCas13b, LbaCas13a, or a PsmCas13b. In some embodiments the CRISPR enzyme is a Cas12a enzyme.

In some embodiments, the sensing agent is connected to a solid surface support. In some embodiments, the sensing agent is attached to the support by a linker. In some embodiments, the linker is an oligonucleotide linker or a peptide linker. In some embodiments, the linker comprises dsDNA, ssDNA, RNA, or a combination thereof. In some embodiments, the linker comprises DNA. In some embodiments the linker comprises modified nucleotides. In some embodiments, the linker is a cleavable linker. In some embodiments, the linker comprises a recognition motif or a cleavage site for an enzyme. In some embodiments the cleavage site is a Cas12 or Cas13 cleavage site. In some embodiments the cleavage site is a Cas12 cleavage site and comprises the sequence TTA or repeats or TTA. In some embodiments the cleavage site comprises a Cas13 cleavage site and comprises the sequence AU, UC, AC, or GA (described in Gootenberg et al., Science (2018) 360:6387 pages 439-444). In some embodiments, bonds such as protease-sensitive (peptide) linkers. β-glucuronide linkers and other common antibody drug conjugate enzyme or chemically cleavable bonds may be used to immobilize probes and sensing agents on solid surfaces to be cleaved by an analyte or activated sensing agents. In some embodiments, the linker is an inactivating linker. Upon cleavage of the inactivating linker the sensing agent is activated.

In some embodiments, the sensing agent comprises a Cas12a CRISPR complex.

Cas12a CRISPR complexes exhibit preferences in their trans-cleavage activity allowing an increase in signal to noise ratios using methods described herein or other methods known in the art. In some embodiments, the Cas12a CRISPR complex has a preference to recognize and/or cleave the nucleotide sequence TTA or TTA repeats.

In some embodiments the activating step comprises cleaving the sensing agent from a support, thereby activating the sensing agent. In some embodiments the activation comprises releasing or detaching the sensing agent into solution.

In some embodiments the surface support comprises a paper or polymer flow strip, a bead, a sensor surface, or a microfluidic channel. In some embodiments, the surface support can be in a cartridge. The cartridge can include a microfluidic cell. The microfluidic cell can include at least one of: (a) a magnetic microstirrer, (b) a plasmonic vortex mixer, or (c) a flow-inducing device. The microfluidic cell can include the magnetic microstirrer, and the system can further include a stage configured to removably engage the cartridge and facilitate alignment of a light path of the interrogator photonic circuit and a light path of the cartridge, in which the stage includes a transmitter configured to power the magnetic microstirrer. The flow inducing device can be an absorptive pad or a microfluidic capillary pump. The microfluidic cell can include at least one of: (i) a protein, (ii) a reagent, or (iii) a rinsing fluid. The microfluidic cell can include at least one microfluidic channel, in which a wall of the channel has an amplifier enzyme or sensing agent attached thereto. In some embodiments, the microfluidic channel transports analyte to a detector.

In some embodiments, the sensing step comprises a sensing probe. A probe is an agent that when cleaved/activated creates a detectable signal such as DNA probes with quencher-fluorophore groups that create a fluorescent signal when cleaved. Probes can be activated by sensing targets or sensing agents.

In some embodiments, sensors for readout of signal may include digital cameras via fluorescent or colorimetric readout, photonic ring resonators and other waveguide devices for surface evanescent field readout and transistors/electrodes for electrochemical readout. The waveguide can include an optical ring resonator or an unbalanced Mach-Zehnder Interferometer. The probe can include an optically active component. The optically active component can be a plasmonic nanoparticle, a gold nanoparticle, a quantum dot, or a fluorophore. The probe can include a silicon particle. The probe can include a magnetic particle. The magnetic particle can include iron-oxide. In some embodiments the photonic waveguide is on a sensing chip or fiber. The sensing chips or fibers may be made using silicon, silicon nitride, silicon dioxide or any other commonly used waveguide materials. In some embodiments, the sensor chiplet is adapted to perform a labeled and label free biosensing tests. In some embodiments, the sensor chiplet performs biosensing via in plane light propagation through waveguides. In some embodiments, the sensor chiplet performs biosensing via reflections (such as Surface Enhanced Plasmon Resonance) or other out-of-plane interactions.

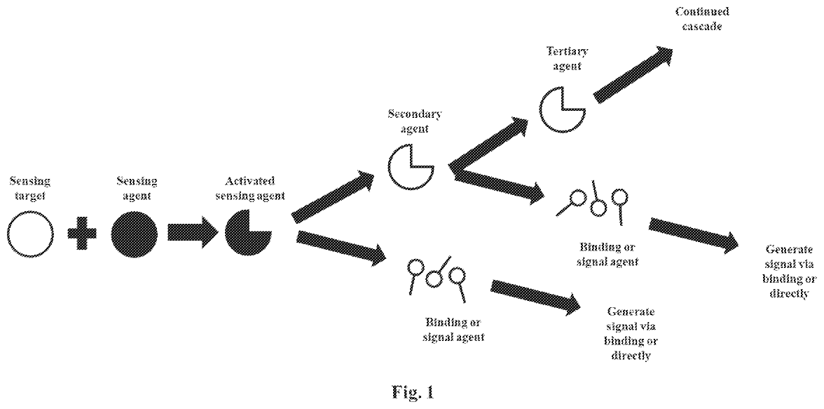

In some embodiments, primary sensing agents (analyte sensing agents) are activated by the presence of the sensing target, (illustrated in the example in FIG. 1). Then the analyte sensing agent may activate one or more secondary sensing agents, which either generate the signal or activates one or more tertiary sensing agents which generate the signal or go on to activate quaternary signal agents and so on. At each step, amplification may be achieved if one agent goes on to activate more than one agent in the next generation. For example in FIG. 1, the detection of a single sensing target by the primary sensing agent results in multiple activated secondary sensing agents that may be detected directly or used to further activate subsequent sensing agents. At each step including the initial sensing agent step, a signal may be generated (for example the sensing agent may be capable of cleaving DNA which activates secondary agents that are immobilized on a solid surface using a DNA linkage in parallel to cleaving DNA probes attached to a sensor surface or DNA probes with quencher-fluorophore groups that create a fluorescent signal when cleaved).

In some embodiments, for example the biological sample can be saliva suspected to contain a viral DNA. A first Cas12a complex (primary sensing target) can be designed with a cognate guide RNA comprising a first spacer RNA complementary or partially complementary to the viral target DNA. The designed Cas12a complex can bind the viral target DNA and is thereby activated to cleave other DNA oligonucleotides that are in the vicinity. A second designed Cas12a complex with a cognate guide RNA comprising a spacer RNA complementary or partially complementary to an unrelated DNA sequence can be attached to a surface support with a DNA oligonucleotide linker. The activated first Cas12a is then brought in close proximity to the second Cas12a complex attached to the surface and binds and cleaves the DNA oligonucleotide linker, thereby releasing the second Cas12a complex into solution. The released second Cas12a complex can be flowed in a microfluidic chip to encounter more attached enzymes, and catalytically bind and cleave further complexes off the support, thereby creating a cascading amplification. The plurality of released complexes can be sensed directly with a detector or can react with a signal agent, releasing a reaction product and the reaction product can be sensed by the detector. The detector signal can then be tallied and the presence of target biomolecule can be confirmed. In some embodiments the released complexes can be sensed by a binding agent, such as an antibody directly bound to the detector or attached to a reporter probe that can be sensed by the detector. The detector signal can then be tallied and the presence of target biomolecule can be confirmed.

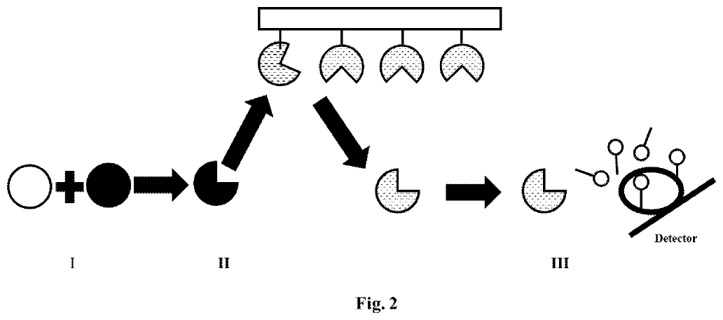

In some embodiments, the secondary or tertiary sensing agent is a cleaving agent (e.g., DNA-ase, HRP-ase) and may cleave reporter probes (e.g., RDA/DNA fluorophore/quencher probes) that then create a fluorescent signal or another detectable signal. The probes may be functionalized onto a surface as shown in FIG. 2 (e.g., gold nanoparticles attached to a waveguide or other sensitive surface with a cleavable linker) or they may be in solution. The reporter probes may themselves, once cleaved from a surface or in solution, diffuse to and bind to a sensor generating a signal or to a tertiary sensing agent, thus activating it.

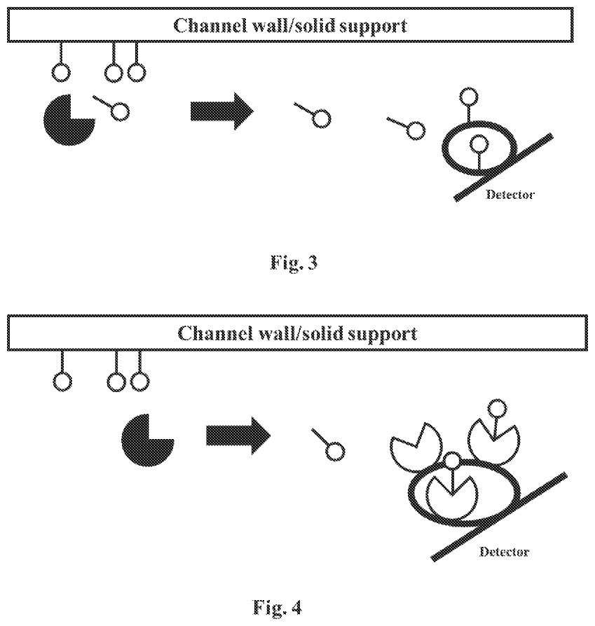

In some embodiments, the secondary sensing agent is a binding agent and may bind to a sensor to generate a signal, as shown in FIG. 3, or it may bind to a tertiary sensing agent, thereby activating the tertiary sensing agent. In some embodiments, the tertiary sensing agent may be a cleavage agent or it may be a binding agent, acting in the same way as the secondary sensing agent described above. In some embodiments, the amplification of the signal can be continued as a chain reaction with as many steps as needed. An example of such a system for sensing RNA or DNA with Cas12 or Cas13 resulting in a signal amplified by cascading amplification, can be seen in FIG. 2.

In some embodiments, the signal is sensed by a sensor. In some embodiments, the sensor is a photonic sensor. Disclosed herein, in one embodiment are photonic biosensors, utilizing cascading amplification. As illustrated for example in FIG. 1, in cascading amplification a system is setup whereby one or more initial sensing agents (analyte sensing agents) are activated by the presence of the sensing target.

In some embodiments the sensing agent may also be activated by a signal such as light (for example, light activated cleavage of the sensing agent from a solid surface), vibration or temperature changes (for example, disassociation of biotin-SA intermolecular bond at high temperature may be used to detach a sensing agent from a solid surface, thus making it active and capable of activating a secondary sensing agent or creating signal at a sensor), instead of a chemical or biochemical analyte.

In some embodiments, the sensing agent (e.g., a CRISPR Cas12 or Cas13 complex for sensing DNA or RNA sequences) may be in solution and is activated specifically by the analyte (e.g., DNA or RNA). The activated sensing agent then activates via cleavage or binding a secondary sensing agent which can be in solution or is attached to a surface (such as a paper flow strip, a bead or a microfluidic channel wall). The secondary sensing agent may also be in solution but is inactivated by the presence or absence of a molecule or complex that may be removed or attached by the activated sensing agent. The secondary sensing agent may be another cleaving or activating agent, or it may be a binding agent.

In some embodiments, a binding agent or probe is located on a detector (e.g., an antibody or protein) and may preferentially capture a cleaved reaction product resulting from an activation step, resulting in a detectable signal as shown in FIG. 4 (e.g., cleaved product labeled with high contrast particle). A secondary o tertiary sensing agent or probe that is attached to a sensor surface may be an enzyme or another complex or molecule that creates an optical, magnetic, electrical or other detectable signal when the primary or secondary sensing agent interacts with it.

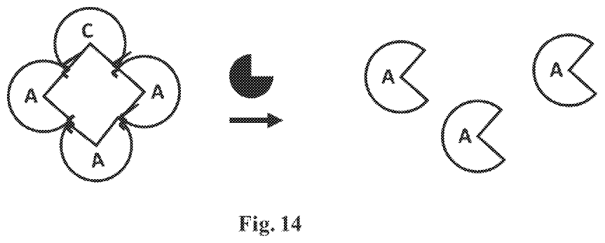

In some embodiments, as illustrated in an example shown in FIG. 13, the sensing agents may act as binders between two or more other molecules such that, upon activation, it creates an activated complex (e.g., an enzyme). In a non-limiting example, two pieces of an enzyme may become a holoenzyme and activated when they bind to an antibody; the activated enzyme may then go on to cleave secondary, tertiary, etc. sensing agents as described above creating a signal that may permit detection of the antibody.

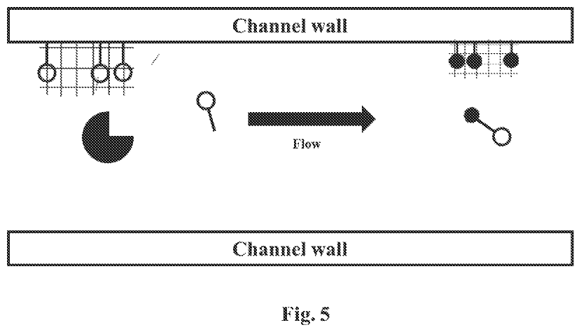

In some embodiments, the cascading amplification reaction occurs in a reaction chamber. In some embodiments, the chamber where the cascading amplification reaction occurs can be optimized to enhance amplification. In some embodiments, the chamber comprises a high surface area system. In a nonlimiting example, high surface area 3D structures like nitrocellulose pads or mesoporous silica may be used to immobilize agents (e.g. enzymes), which are then cleaved of by activated sensing agents after the analyte is detected, as illustrated in FIG. 5.

In some embodiments, the analyte solution may be incubated in a one-pot reaction or it may be flowed through high surface area systems, simple channels, or flow assay strips, allowing for physical separation of events as follows: Sensing agent is mixed with sample suspected to comprise an analyte, upon presence of the analyte of interest in the sample the primary sensing agent is activated. The activated primary sensing agent is then flowed through the system where it encounters and reacts with dried secondary sensing agents attached to a surface. Upon reaction with the secondary sensing agents, the activated primary sensing agent cleaves the secondary sensing agents from the surface such that they are dissolved in the flowing liquid. As the liquid passes to the next section of the flow assay it encounters further dried sensing reagents (probes or tertiary agents) which can react with the secondary agent to create a color change or other detectable signal.

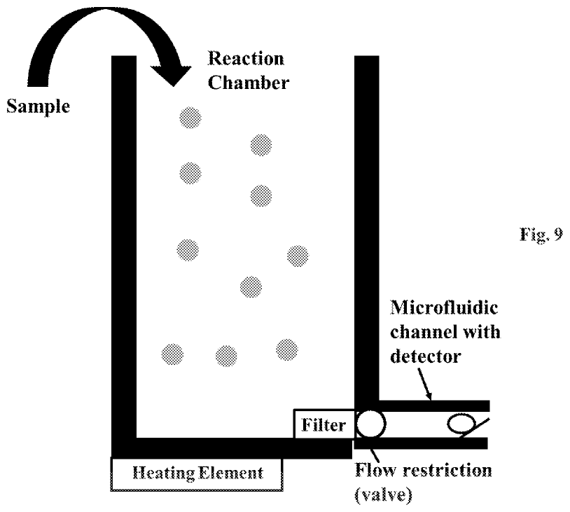

In some embodiments, the sample can be flowed into one or more bulk chambers where the amplification reaction takes place. Then, as seen in an example shown in FIG. 9, either via valve or a continuous flow or pumped or vacuum small quantities of supernatant can be flowed into detection chambers or into microfluidic channels that flow over sensors. This allows the reaction to take place in a bulk high surface area, high volume chamber, but the output signal can be continuously monitored until a positive or negative signal can be established.

In some embodiments, non-biological and non-chemical agents may be used. For example, electrical or optical components such as transistors or optical resonators may be used to as agents which go on to activate other agents. In one nonlimiting example, an agent may activate a transistor either via cleavage of particles from its surface or by binding to the surface, which in turn may change the current in a magnetic circuit releasing agents into a solution that are functionalized onto magnetic beads.

In some embodiments, the test cartridge is disposable. In some embodiments, the sensor chiplet on the sensor tip is disposable. The disposable tip-based biosensing chip may include multiple optical components (e.g., waveguides or optical fibers) that can be used to multiplex different tests (e.g., immunoassays, viral RNA/DNA, etc.) on the same test sample (e.g., in the same well). Redundant testing (e.g., for the same virus) may increase sensitivity and/or specificity, while multiplexing tests for multiple pathogens (e.g., COVID-19 and flu) and/or multiple patient samples may be advantageous for facile widespread testing. Coupled automated liquid handling could permit these redundant and/or multiplexed tests to run more precisely and efficiently.

In some embodiments, a signal can be generated at more than one step of the amplification cascade. This signal may be read via the same or different methods as the final signal. For example, the sensing agent (e.g., a CRISPR Cas complex) may cleave DNA or RNA linkages (e.g. after finding a target RNA or DNA), some of which, when cleaved release or activate a secondary sensing agent (e.g., a peroxidase or other enzyme bound to a porous medium or microfluidic channel surface with ssDNA via biotin-streptavidin or other linkage between the DNA and the peroxidase enzyme), while others create a detectable signal when cleaved (e.g., a gold nanoparticle bound to an optical resonator with ssDNA). Such dual function can serve both to improve sensitivity and specificity but also to monitor the reaction and provide controls.

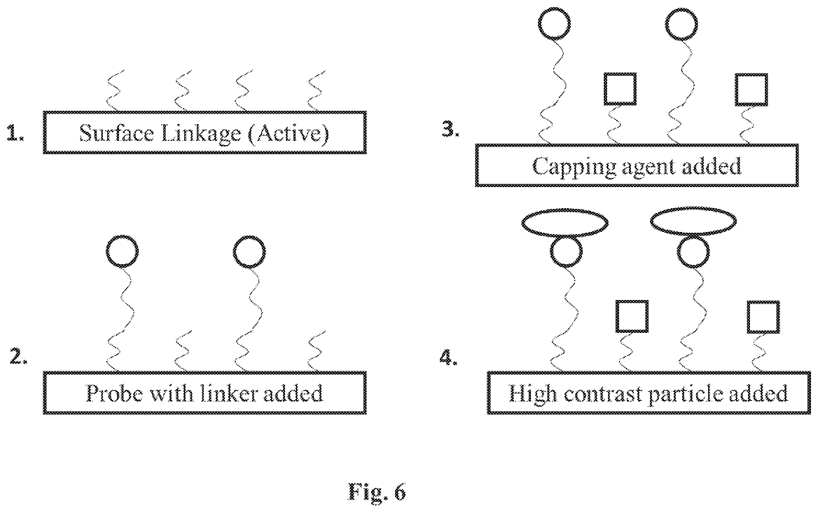

Another aspect of the present disclosure, as seen in an example shown in FIG. 6, relates to controlling surface functionalization of the sensing agents and probes that permits cascading amplification. Surfaces may be modified with any common surface modification group (e.g., amines or carboxylic acids) such that probes may be attached. In some embodiments probes can be attached by a linker. When modifying or functionalizing a surface, the agents or high contrast particles attached to the other side of the linkage may also interact with and/or be bound to the functionalized surface. In some cases, this may be prevented by first linking the probe to the surface. In one example, DNA/RNA/oligomer of another polymer/molecule/particle, (e.g., the surface is carboxylic acid functionalized, DNA with an amine group on one side (5′ or 3′ end), biotin on the other 5′ or 3′ end) can be attached to the surface, then a capping agent may be flowed over the surface to cap the unused functional groups (i.e. an amine to cap the carboxylic groups), then the agent can be flowed over the newly attached linkage to connect to the portion that is not connected to the surface. This is illustrated schematically in FIG. 5. For example, in the amine-DNA-biotin case it would be the biotin (e.g., using streptavidin) on the 5′ or 3′ end of the DNA capturing an AuNP high contrast particle. This may limit undesired interactions wherein a high contrast particle, agent, or enzyme reacts with the functionalized surface (e.g., streptavidin reacts with carboxylic acid) and binds to the surface.

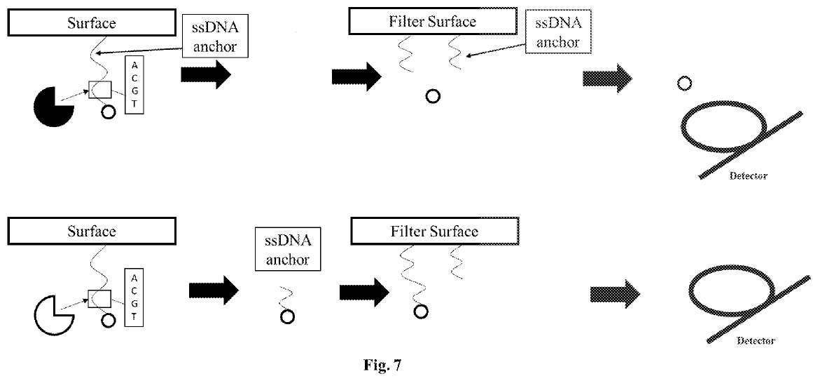

In some embodiments, and as seen in an example shown in FIG. 7 one of the sensing agents may preferentially cleave a specific sequence on a DNA or RNA oligonucleotide that is used as an agent or is used to immobilize agents onto a surface or inactivate them. Once cleaved in the specific position on the sequence a given specific oligonucleotide design may result in a preferential sequence length or barcode that can either be used for detection of that barcode downstream or as a tag for the agent that it is attached to. Additionally, if the oligonucleotide is designed to be cleaved by the sensing agent such that the part of it that is left on the secondary agent (which was immobilized or inactivated with this oligonucleotide as a linkage) is very short or does not contain some specific barcode or sequence, then any such oligonucleotide linkage that is cleaved inadvertently (e.g., via side reactions from other enzymes or chemicals in the sample or due to thermodynamic degradation of the linkage or other sources of noise) may predominately leave a much longer oligomer attached to the secondary agent when compared to the specifically cleaved oligomer left after proper sensing agent cleavage. This longer sequence or barcode can be designed such that it can bind to another ssDNA or ssRNA that is surface immobilized, thus allowing the filtering out of secondary agents that have become activated or have been cleaved off a surface my means other than the sensing agent, such as other nucleases in the sample. As shown in FIG. 7, this may help distinguish the presence of the activated sensing agent from other sources of cleavage or activation. The cleavage may not need to be completely specific to the desired sequence length or barcode to reduce signal noise, as even semi-specific cleavage would increase the concentration of secondary sensing agents that have been cleaved such that they carry the desired barcode or sequence length vs. random sequences within the system. In some embodiments, the short length of the correctly cleaved barcode allows it to avoid capture by an immobilized ssDNA filter, if the sensing agent is not 100% sequence specific and occasionally cleaves at a different point in the oligonucleotide sequence, then some signal will be lost as secondary sensing agents cleaved by the sensing target may be captured and filtered out along with those that have been cleaved by sources of noise. However, the signal to noise ratio is increased as the portion of secondary sensing agents that are correctly cleaved may be the only source of signal downstream, thus avoiding a false positive signal.

In some embodiments, another sensing agent or sensing probe could be held onto a surface such that the DNA or RNA oligonucleotide that is still attached to the cleaved component is either a specific barcode sequence or a specific length (e.g., if the original DNA was cleaved at a specific sequence). Alternatively, the sequence may be cleaved in a place where it will no longer have enough oligonucleotide sequence exposed to be readily captured, such that anything that is cleaved by the sensing agent will be specifically allowed to flow through the system, while anything removed from the surface due to a non-specific cleavage will be filtered out (e.g., its longer DNA strand hanging will be captured by a surface featuring sites for capturing DNA).

In some embodiments, Cas12a CRISPR complexes may be used as sensing agents or secondary sensing agents as they exhibit preferences in their trans-cleavage activity allowing an increase in signal to noise ratios using methods described herein or other methods known in the art.

In some embodiments, the cleavage site of the oligonucleotide linker is designed to such that the cleavage will occur near the solid support surface rather than near the agent when it is initiated by an activated sensing agent. After specific cleavage of the linker by the sensing agent the ssDNA or RNA tail on the released or activated sensing agent will be longer than from random cleavage events. Thus, the resulting longer linker can be used to capture the agent as a binding agent on sensor surfaces or to attach it to other agents or probes, leading to a signal. In a nonlimiting example, high contrast nanoparticles may be attached to a solid surface using an ssDNA or RNA oligonucleotide linker. When this particle is cleaved off the surface by an activated sensing agent (such as a CRISPR Cas12 or Cas13 complex activated by RNA or DNA), it retains part of the oligonucleotide linker. This oligonucleotide can be designed for the upper half (5′ end or 3′ end) to be a used as a barcode that allows it to later be bound to another solid support that has an oligonucleotide with a complimentary or partially complementary nucleotide sequence attached. This solid support may be a sensor surface (e.g., a waveguide or plasmonic surface) which generates a signal from the binding event. Subsequently, the validity of the signal (vs. non-specific binding) may be confirmed by exposing the newly formed double stranded bond to a DNase enzyme that preferentially cleaved double stranded DNA.

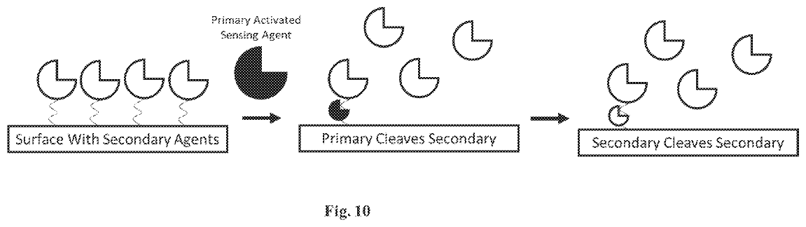

In one embodiment, as seen in an example shown in FIG. 10 additional amplification may be achieved by designing the linkage that immobilizes or inactivates agent A cleavable or removable by agent A as well as the agent in the previous step of the amplification cascade. Thus, after the previous agent activates even one agent A, that agent can active more As and so on, leading to a cascading amplification. To achieve the surface immobilization or inactivation of agent A, the agent may be inactive while it is being immobilized or inactivated by binding to a linker (such as a DNA oligonucleotide or other oligomer or protein, or molecule). To achieve this, the temperature may be set such that A is not active (for example outside the active temperature range for an enzyme), or the pH or other conditions may be varied. Once all As are immobilized or inactivated they may not autoactivate until an agent from a previous step in the cascade activates/cleaves them. A nonlimiting example of an agent A may be an DNase capable of cleaving ssDNA that is surface bound by a ssDNA or a HRP using a linkage that it can cleave. In the example of CRISPR Cas12 acting as a sensing agent for DNA, it can cleave DNase (for example) from a surface, as well as DNA probes from a sensor, and the DNase can cleave more DNAase from the same surface as well as probes from a sensor.

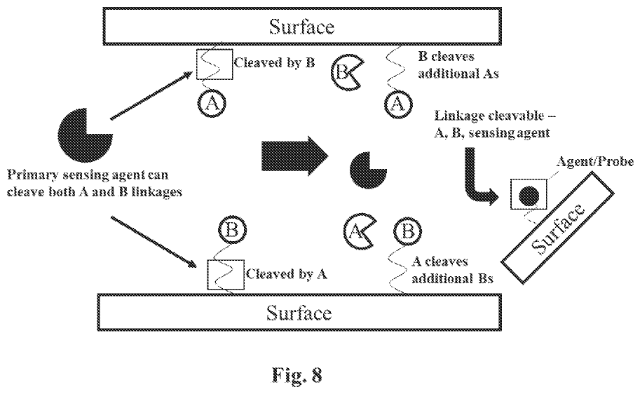

In another implementation, as seen in an example shown in FIG. 8, additional amplification may be achieved by designing the linkage that immobilizes or inactivates agent A cleavable or removable by agent B, and the linkage that immobilizes or inactivates linkage B cleavable or removable by agent A, as shown in FIG. 8. Both linkages or one of them may be cleavable or removable by the agent in the previous step of the cascade (e.g., the sensing agent or secondary or tertiary etc. agent). Once one of the agents A or B is activated, it activates one or multiple agents B, which activate agent As and so on, leading to a large amplification. This is favorable because agent A may be attached to a surface or inactivated in the absence of agent B, and agent B in the absence of A. Then they can be exposed to the same fluid in a microfluidic cell or in a bulk reactor such that an agent from the previous step of the amplification cascade can cleave or activate an A or B agent and start the amplification. For example, a CRISPR sensing agent may cleave off surface agents A and B which are surface bound with different DNA oligonucleotides. A's oligonucleotide is designed to be cleaved by B (a restriction enzyme aimed at a sequence contained in A's oligonucleotide) and vice versa. Thus when a CRISPR sensing agent becomes activated it cuts an A or B and starts the amplification.

In some embodiments, molecules, complexes, proteins, etc. may be generated at some step in the cascade. For example RNA or DNA may be generated via transcription, reverse transcription or copying, or proteins such as enzymes may be created via translation, or easily detectable molecules like glucose may be generated via catalysis or synthesis using other molecules in the solution.

In some embodiments, a surface effect-based sensor, such as an optical resonator is used to detect the signal. In some embodiments, a DNA oligonucleotide is attached to the surface of the waveguide with a oligonucleotide sequence at the base that codes for a complementary oligonucleotide sequence on a binding agent. This will allow the binding agent to bind close to the sensing surface, thereby improving signal. If a sandwich assay is used (capture agent attached to the surface, captures a binding agent, which captures another capture agent) it may be set up this way: First a ssDNA or RNA is attached to the surface of the sensor, then a capture agent like an antibody is attached to the top of the DNA or RNA oligo, then that capture agent binds an analyte or an activated sensing agent or a secondary/tertiary/etc. agent that has been cleaved or activated, called C for the purposes of this example. Then another agent, called agent D, that is present in solution and activated (it may not require activation) binds to the binding agent that is connected to the surface via the linkage described above. In some embodiments. D has a high contrast particle, like an AuNP attached to it via a DNA or RNA oligonucleotide, such that it binds to the DNA or RNA oligonucleotide that is attached to the surface of the sensor, bringing the high contrast particle near the surface. This binding can be designed (via sequence choice) to be weak, meaning that it is unlikely to happen or to stay bound if it happens unless D is also bound to C.

In some embodiments, agents may act as barriers to flow or diffusion either as membrane made of agents A crosslinked (e.g., DNA origami) or otherwise connects such that it filters or prevents flow in a microfluidic channel or porous medium. Then an activated sensing agent or an activated agent from a previous step of the cascade (e.g., CRISPR Cas12) may cleave or in another way degrade the membrane/pore blocking complexes made of agents A. This then opens up channel(s) for the flow or diffusion of particles, agents, molecules and other analyte into a different portion of the microfluidics. This can also be done in the context of a lateral flow assay. Once flow of agents etc. starts, it can then lead to secondary events downstream that activate more agents or lead to signal. For example, enzymes that were previously prevented from passing through the membrane or pores that were clogged with Agent A may flow to a sensor and cleave a probe from the sensor or binding agents may flow to a sensor and bind.

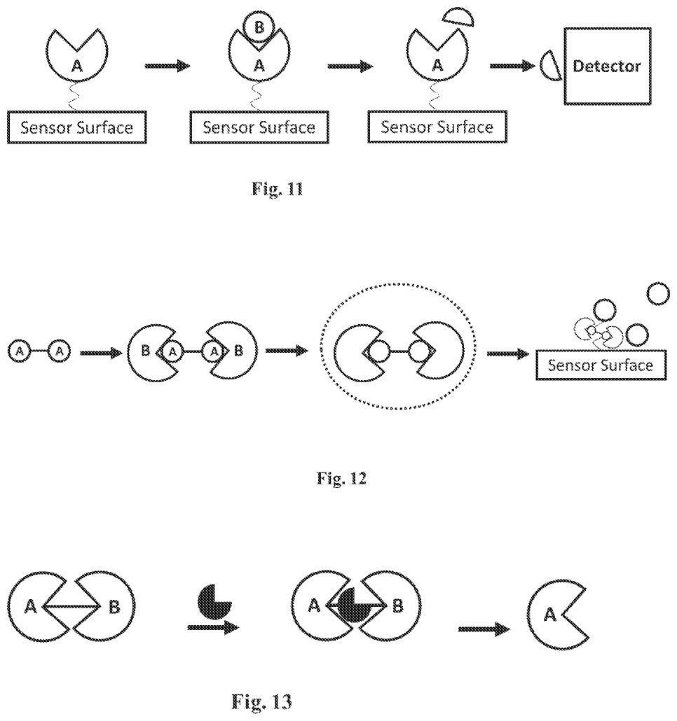

In some embodiments, a surface is functionalized with agent A. For example FIG. 11 shows an example where a surface is functionalized with agent A. Agent A may then capture and/or cleave agent B, resulting in a detectable signal. FIG. 12 shows how a complex C may cleave probes from a sensor or agents from a surface. FIG. 14 discusses an example wherein an activated sensing agent (agent B) may cleave linkages that connect agents (agents A and/or C which may feature another complex/molecule/particle). Cleavage of this group of connected agents may result in activated agents (activated agent A for example).

### Binding Assays

In some embodiments, an analyte can be detected through binding to a molecule immobilized on or near a waveguide. For example, binding of antigens to antibodies that are immobilized on or near a waveguide can be detected by an integrated photonic sensor. The evanescent field emanating from the waveguide is used to then sense a refractive index change due to the presence of antigen (for example an activated sensing agent) after binding. In another embodiment, biosensing is performed by a biological marker (e.g., virus antigens, antibodies, etc.). The biological markers may be immobilized at or near the waveguide. In some embodiments, whole pathogen detection is performed. The pathogen may be bound to a waveguide by functionalizing the waveguide with antibodies that capture the pathogen. However, because the refractive index of a virus, for example, is in the range of 1.4-1.5 and water is 1.33, it can be hard to detect a single viral particle. To increase the signal, an optically active component may be attached to the pathogen. In some embodiments, a plasmonic particle or other complex with strong optical properties may be attached to the pathogen by functionalizing the nanoparticle with antibodies for the pathogen. The pathogen may be bound to a waveguide by functionalizing the waveguide with antibodies that capture the pathogen. In some examples, RNA or DNA is first functionalized with a reporter probe, then it may bind to conjugate DNA or RNA attached to the waveguide. The reporter probe may have a sequence which precisely binds the DNA or RNA (single strand). When the reporter probe is away from the waveguide, the binding site is therefore closed off. When the reporter probe connects to the sensing target (e.g., viral DNA) it unfolds, and the binding site is revealed. The biological markers may be in solution and bind to the waveguide in any number of ways. The waveguide may then detect the refractive index change due to the presence of the biological marker at or near the waveguide. Alternatively, if the biological marker is optically active in the region the waveguide operates at, light intensity may simply be measured after passing through the waveguide.

### Cleavage Assays

In some embodiments, a component of a sample can be detected by directly or indirectly resulting in a cleavage reaction which is detected by a sensor chiplet. In some embodiments the cleaving agent (for example an activated sensing agent) cleaves a reporter probe from a waveguide. In one example, a waveguide (e.g., associated with a ring resonator) is functionalized to immobilize reporter probes (e.g., RNA strands). Next, a cleaving component (e.g., a CRISPR enzyme) that may interact with the reporter probes and a sensing target of interest may be combined with analyte carrying the sensing target. In some embodiments, the testing mechanism used by the probe apparatus utilizes A—waveguide, B—ring resonator, C—functionalized nanoparticles (e.g., reporter probe with optically active component), and D—sensing agents capable of cleaving the nanoparticles from the ring resonator. In one example, a waveguide (e.g., associated with a ring resonator) may be functionalized to immobilize reporter probes (e.g., RNA strands). These reporter probes may be linked to an optically active component (e.g., plasmonic nanoparticle, quantum dot, molecule, etc.) to enhance their optical effect on the waveguide and/or for downstream detection. Next, a sensing agent or cleaving component (e.g., a CRISPR enzyme) that may interact with the reporter probes and a sensing target of interest (e.g., virus RNA or DNA) may be combined with analyte carrying the sensing target (e.g., viral RNA from patient sample introduced into test cartridge). In some cases, the sensing agents are activated to cleave the functionalized nanoparticles only if they encounter biological material associated with a positive test result (e.g., viral RNA). The cleaving component may be activated, thereby indiscriminately cleaving the functionalized nanoparticles. In some examples, if the reporter probes attached to the waveguide are removed, an optical change in the system may be detected in various ways. In one example, cleavage of the probes from the waveguide may result in a change in the refractive index of light guided within the waveguide; this change in refractive index may be detected using various spectroscopic techniques (e.g., resonance, interference, or absorption, etc.). Additionally or alternatively, the optically active component (e.g., plasmonic nanoparticle, quantum dot, molecule, etc.) attached to the reporter probes may be cleaved along with the reporter probes. The presence of these cleaved optically active components may be detected downstream from the waveguide using various techniques. Other known techniques for facilitating interactions between the waveguide and sensing targets, reporter probes, biological markers, pathogen, etc. (e.g., toehold switch) may be implemented in addition to or as an alternative to the described techniques. In some embodiments, the cleaving component is designed to be activated only when it detects the sensing target of interest. Once the cleaving component is activated, it cleaves probes from the sensing surface, leading to a detectable signal (via resonance, absorbance, interference resistance changes or other detectable changes near the sensing surface). In some embodiments, the cleaving component binds to the sensing target of interest. The cleaving component may be activated, thereby indiscriminately cleaving both the sensing target and immobilizer probes. Various cleaving components (e.g., CRISPR enzymes activated by target RNA, or other enzymes activated by an analyte of interest) that may cleave the reporter probes, removing them from the surface, when an analyte of interest binds to or is otherwise detected by the cleaving agents in solution.

In some embodiments, the probes are engineered to enhance the signal generated by cleavage events, which is distinct from other techniques where binding of analyte to the surface directly generates a signal. The readout maybe done by immobilizing the probes on the surface of waveguides, such that the evanescent field interacts with the probes, but any surface method or any combination of surface methods (e.g., electrical and/or optical) may be used including transistors, nanopores, surface plasmon resonant thin films or particles, surfaces used for SERS spectroscopy, or electrical impedance (e.g., resistance) based sensors. In some embodiments, a high contrast cleavage detection system, where there is both a cleaving component that is either the analyte of interest or has a specific detection mechanism for the analyte of interest, and a solid state probe that is functionalized onto a sensing surface (e.g., a waveguide, plasmonic thin film, etc.), is used.

In some embodiments, the cleavage event is caused by the analyte of interest or may be facilitated via a chemical in solution and/or from electromagnetic radiation (e.g., UV light). The method may be used directly to detect any effect that causes the probe removal; this includes light, heat and other changes in the environment generally or locally that can cause the probe to detach. In a nonlimiting example, probes may contain UV cleavable linkages or heat-disassociated bonds. For sensing analytes in solution that are exposed to the surface, the cleavage event may be activated by a chemical or enzyme associated with the sensing target. In one nonlimiting example, the cleaving component may be an enzyme (e.g., CRISPR, a Toehold Switch RNA detection produced enzyme or protein) that may cleave reporter probes (e.g., RNA strands) immobilized on the surface of an electronic, magnetic, MEMS, optical, or optoelectronic device. The cleaving component may be activated when it detects the sensing target of interest in solution, thereby cleaving the immobilized reporter probes. In some cases, the immobilized reporter probes consist of an optically-active and/or conductive or magnetic component, which may facilitate detection of this cleavage event (e.g., via the optical signal or a change in resistance at an electrode described above). This cleavage may be sensed directly where it happens (e.g., by a change in response of a ring resonator/optical waveguide where the reporter probes were immobilized prior to cleavage) or the cleaved products (e.g., the cleaved reporter probes migrate away from the surface for detection elsewhere in the system). The cleaved products may migrate to and bind to a sensing surface via diffusion or mixing.

In some examples, the cleaved product may be designed for strong binding affinity to the sensing surface (e.g., surface functionalized gold particles functionalized with biotin designed to bind to sensing surface functionalized with Streptavidin). This method may also be used to determine or sense activity or reaction kinetics associated with a biomolecule or enzyme even if the reaction is reversible. For example, if the surface is functionalized with an agent the biomolecule reacts with, a binding event associated with this reaction may be detected (e.g., via optical resonance shift etc.), and if the complex falls apart or is broken, this can be detected as a cleavage. The contrast can be increased by labeling the component that is added from solution using a gold nanoparticle or otherwise optically/magnetically/electrically active label that interacts strongly with the surface. Additionally or alternatively, various enzymes may be attached to various surfaces and their activity may be monitored separately using the optical and/or electronic interactions described above. For example, an optical system may include multiple ring resonators where each ring resonator may be functionalized with a different enzyme (e.g., CRISPR Cas12, Cas13, etc.). These various cleaving components may be designed to be activated only when they are exposed to their specific sensing target of interest. Once activated, each cleaving component, tethered to the sensing surface, may cleave only cleave probes in its direct vicinity. This may lead to a change in the response (e.g., plasmon resonance optical readout or electronic transistor readout) of only the surface where the cleavage occurred. This may permit different regions or different surfaces to detect different analytes in the same sample without any interference and without the need for any microfluidic or other physical separation. In the nonlimiting example of RNA sensing with ring resonators, different resonators on a chip may be functionalized by a CRISPR enzyme carrying a different crRNA sequence, allowing each ring to become a sensor for that specific sequence when all the rings are exposed simultaneously to the same analyte. This can work with any version of the High Contrast Cleavage approach described.

In some embodiments, instead of attaching different enzymes or other cleaving agents with different target analytes to different sensing surfaces, the sample fluid may be split up into separate chambers, each containing a different cleaving agent (in a dried state or added via a different fluid input channel/port) with a different target analyte. This allows testing of the same sample for different analytes in parallel without interference. It may also be arranged in a serial fashion, where the sample flows first over a sensing surface where the microfluidic chamber contains the first cleaving agent, then flows into a chamber with the second cleaving agent, and so on (e.g., each chamber containing 1 or more sensing surfaces with cleavable probes). Using the two above described techniques (separate optical system with distinct enzyme, splitting sample fluid) may be useful for both redundant testing (e.g., for the same virus) by increasing sensitivity and/or specificity and multiplexing tests for multiple pathogens which may be advantageous for facile widespread testing.

In some embodiments, rather than probes, secondary cleaving agents may be surface bound or otherwise “locked/inactivated” by a cleavable linkage and removed/unblocked/activated via cleavage by the primary cleaving agent when an analyte is sensed. This technique may be useful as an amplification technique as a small cleavage event associated with a small amount of the sensing target of interest could cascade into the cleavage of a large number of reporter probes that may be more easily detectable by the optical, electronic, magnetic, MEMs, or optoelectronic device. In other words, this creates an amplification whereby one primary cleaving agent (e.g., CRISPR), after becoming activated by the analyte of interest, cleaves many secondary cleaving agents, which then go on to cleave probes from a sensing surface. In some cases, cleavage may also occur in solution or on surfaces in a way that either releases/activates secondary cleaving agents (e.g., for cleaving probes from sensing surfaces) or high contrast probes that go on to bind to sensing surfaces.

### CRISPR Assay

In some embodiments, the cleaving component is a CRISPR Cas13 complex which cleaves all nearby RNA upon activation, including the RNA reporter probes immobilized on the waveguide. A waveguide may be functionalized using standard methods with DNA or RNA strands. These strands may be linked to a plasmonic (e.g., gold) nanoparticle, quantum dot or another molecule to enhance their optical effect on the waveguide. A CRISPR enzyme such as Cas12 (for DNA detection) or Cas13 (for RNA detection) carrying the relevant sequence may be combined with analyte carrying the RNA or DNA of interest. When the CAS protein binds the RNA or DNA of interest it may be activated and used to cut multiple RNA or DNA strands nonspecifically. If the DNA or RNA strands attached to the waveguide are cut, the effect on the guided light within the waveguide can be detected using one of the methods described above (resonance, interference, or absorption). See Integrated Micropillar Polydimethylsiloxane Accurate CRISPR Detection (IMPACT) System for Rapid Viral DNA Sensing. Kenneth N. et al. for a similar approach.

In some embodiments, after a sensing target is identified, the CRISPR cleaving component may cleave a cluster of enzymes connected with an RNA or DNA scaffold. These enzymes may become activated and may cleave probes from the photonic waveguide. In some cases, they may not be enzymes but instead some type of particle that binds to the waveguide. This binding changes the local refractive index. The binding site is therefore hidden when they are connected to the cluster. Thus, the binding site may only be opened when the particle is cleaved. The processes above describe several possible sensing techniques using a photonic waveguide, as taught herein. These processes may be further performed with or without common techniques associated with biosensing (e.g., target amplification). Other known techniques for facilitating interactions between the waveguide and sensing targets, reporter probes, biological markers, pathogen, etc. (e.g., toehold switch) may be implemented in addition to or as an alternative to the described techniques.

### Further Biosensing Embodiments

In some embodiments, the target of interest may first be chemically amplified using techniques including but not limited to PCR or RT-LAMP or RPA. In some cases, reverse transcriptase may be used to convert RNA to DNA. This may allow for DNA sensing systems like PCR or CRISPR Cas12 to be implemented. For PCR, the sensing protocol may include emitting light into the analyte using vertical grating couplers or an evanescent field and then observing fluorescent response either using external or on-chip optics and photodetectors. In another aspect of the present disclosure, a chemical reaction on the surface of an optical, electronic, magnetic, MEMs or optoelectronic device may be catalyzed. In one example, a chemical reaction at a waveguide may be catalyzed on a waveguide via an evanescent field associated with the waveguide. In some cases, the chemical reaction may be controlled via integrated photonics (e.g., by toggling the light on and off or switching between different input wavelengths) to activate chemical reactions selectivity (e.g., which reaction, where the reaction occurs, when the reaction occurs, etc.). Additionally or alternatively, reaction kinetics could be further controlled by controlling the intensity and/or wavelength using components such as ring resonators, optical switches, photonic crystals, Bragg gratings, LEDs, and lasers which are capable of introducing and controlling high-intensity light across a range of wavelengths. MEMs components may be fabricated either instead of or in complement to other components in order to control chemical reactions near the surface, induce mixing, induce polymer folding, induce strain in the surface or in polymers attached to the surface etc. In all cases, sensing may be done in parallel or serially as chemical reactions are occurring/being catalyzed/controlled. In one implementation of High Contrast Cleavage Detection, an antibody, antigen or another analyte (which itself may be a complex of the target analyte and another molecule) may act as a bridge to combine two or more separate molecules into cleaving agents which goes on to by an input to the sensing method as described above. Additionally a cleavage agent may be designed with a blocked active site such that the blocking element can disassociate in the presence of the correct analyte or when some change is sensed (pH, temperature, etc.), again working as an input to the sensing method.

### Optically Active Components

In some embodiments, if the reporter probes attached to the waveguide are removed, an optical change in the system can be detected in various ways. In one example, cleaving the reporters from the waveguide may result in a change in the refractive index of light guided within the waveguide; this change in refractive index may be detected using various spectroscopic techniques (e.g., resonance, interference, or absorption, etc.). Additionally or alternatively, the optically active component (e.g., plasmonic nanoparticle, quantum dot, molecule, etc.) attached to the reporter probes may be cleaved along with the reporter probes. The presence of these cleaved optically active components may be detected downstream from the waveguide using various spectroscopic techniques (absorption, photoluminescence, fluorescence, etc.). These reporter probes may be linked to an optically active component (e.g., plasmonic nanoparticle, quantum dot, molecule, etc.) to enhance their optical effect on the waveguide. Further, anything being captured by an antibody may be enhanced by attaching an optically active probe to it.

### Reaction Kinetics

Several methods to increase the likelihood of interaction between the waveguide and analyte containing sensing targets, reporter probes, biological markers, pathogens, etc. are described. In one example, optical trapping (e.g., using strong electric field near waveguide or other photonic structure, similar to optical tweezers) to trap the sensing target at or near the waveguide. Additionally or alternatively, magnetic nanoparticles may be bound to the sensing targets, biological markers, or pathogens of interest. The sensing target, biological marker, or pathogen of interest may then be drawn to the sensing waveguide using a magnetic field applied externally or on the sensor. By binding magnetic nanoparticles to the molecule or pathogen of interest, the complex may then be drawn to the sensing waveguide using a magnetic field, applied externally or via an electromagnet fabricated directly onto the sensor chiplet. This method may be combined with any diagnostic scheme, including those discussed above. Additionally, one or more plasmonic antennas (e.g., a bowtie) may be fabricated on the chip such that local light-induced heating causes mixing via convection.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of.” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items. Use of ordinal terms such as “first.” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in an embodiment of the disclosure.

Unless otherwise defined, all technical and scientific terms used herein have the same meaning as commonly understood by one of ordinary skill in the art to which this disclosure belongs. In the case of conflict, the present specification will control. Although methods and materials similar or equivalent to those described herein can be used in the practice or testing of the present disclosure, suitable methods and materials are described below.

What is claimed is:

1. A method for assaying a biological sample suspected of comprising a target biomolecule, comprising:
contacting the sample with a primary sensing agent, wherein the primary sensing agent is activated in the presence of said target biomolecule;
providing conditions for activating a secondary sensing agent in the presence of said activated primary sensing agent, wherein said activated primary sensing agent comprises a first catalytic activity that results in activation of said secondary sensing agent comprising a second catalytic activity;
providing conditions for detecting a signal agent, wherein said activated secondary sensing agent comprises a second catalytic activity for a reaction that results in detection of said signal agent; and
detecting a quantity of a signal generated by said signal agent, thereby assaying said biological sample for said target biomolecule.

2. The method of claim 1, wherein activation of said secondary sensing agent comprises cleaving a linker binding said secondary sensing agent to a surface.

3. The method of any one of claim 1 or claim 2, wherein said primary sensing agent and/or said secondary second sensing agent is an enzyme.

4. The method of claim 3, wherein said primary sensing agent and/or said secondary sensing agent is a cleavage enzyme.

5. The method of any one of claims 1-4, wherein the target biomolecule is a biological marker.

6. The method of claim 5, wherein the target biomolecule is selected from the group consisting of a dsDNA, a ssDNA, a RNA, a peptide, a protein, an antigen, and a pathogen.

7. The method of claim 5, wherein the target biomolecule comprises a RNA or a DNA.

8. The method of any one of claims 1-7, wherein the biological sample is selected from the group of blood, cord blood, saliva, mucus, tissue, nasal swab, and urine.

9. A method for detecting a target biomolecule in a biological sample, comprising contacting the sample with a primary sensing agent, wherein the primary sensing agent is activated by said target biomolecule;
contacting said activated primary sensing agent with a secondary sensing agent, wherein said activated primary sensing agent catalyzes a first reaction that results in activation of said secondary sensing agent comprising a second catalytic activity;
contacting said activated secondary sensing agent with a signal agent, wherein said activated secondary sensing agent comprises a second catalytic activity for a second reaction that results in detection of said signal agent; and
detecting a signal generated by said signal agent, thereby detecting said target biomolecule in said biological sample.

10. The method of claim 9, wherein the target biomolecule is a biological marker.

11. The method of claim 9, wherein the target biomolecule is selected from the group consisting of a dsDNA, a ssDNA, a RNA, a peptide, a protein, an antigen, and a pathogen.

12. The method of claim 9, wherein the target biomolecule comprises a RNA or a DNA.

13. The method of any one of claims 9-12, wherein the biological sample is selected from the group of blood, cord blood, saliva, mucus, tissue, nasal swab, and urine.

14. The method of any one of claims 9-13, wherein the primary sensing agent comprises a CRISPR enzyme.

15. The method of claim 14, wherein the CRISPR enzyme comprises a Cas12 complex or a Cas13 complex.

16. The method of claim 15, wherein the CRISPR enzyme comprises a Cas12a complex.

17. The method of claim 15, wherein the CRISPR enzyme comprises a Cas13 selected from the group consisting of a LwaCas13a, a CcaCas13b. a LbaCas13a, and a PsmCas13b.

18. The method of any one of claims 9-17, wherein the secondary sensing agent is an enzyme selected from the group consisting of a restriction enzyme, a hydrolase, a peroxidase, a lyase, a ligase, a glutathione S-transferase, and a SpyTag/SpyCatcher.

19. The method of claim 9, wherein the secondary sensing agent has nuclease activity.

20. The method of claim 19, wherein the secondary sensing agent comprises a CRISPR enzyme.

21. The method of claim 20, wherein the CRISPR enzyme comprises a Cas12 complex or a Cas13 complex.

22. The method of claim 21, wherein the CRISPR enzyme comprises a Cas12a complex.

23. The method of claim 21, wherein the CRISPR enzyme comprises a Cas13 selected from the group consisting of a LwaCas13a, a CcaCas13b. a LbaCas13a, and a PsmCas13b.

24. The method any one of claims 9-17, wherein the secondary sensing agent is an binding protein selected from the group consisting of an avidin, a maltose-binding protein, and a chitin-binding protein.

25. The method any one of claims 9-24, wherein activation of said secondary sensing agent comprises cleaving a first linker binding said secondary sensing agent to a first surface.

26. The method of any one of claims 9-24, wherein activation of said secondary sensing agent comprises cleaving an inactivating linker on said secondary sensing agent.

27. The method any one of claims 25-26, wherein the linker comprises ssDNA, dsDNA, RNA, or a combination thereof.

28. The method of claim 27, wherein the linker comprises an oligonucleotide or a modified oligonucleotide.

29. The method any one of claims 25-28, wherein the linker comprises a cleavage or recognition site for the primary sensing agent.

30. The method of claim 29, wherein the recognition or cleavage site comprises a TTA sequence or a TTA sequence repeat.

31. The method of claim 29, wherein the recognition or cleavage site comprises a sequence selected from the group consisting of AU, UC, AC, and GA.

32. The method of claim 12, wherein the linker is a peptide linker.

33. The method of claim 9, wherein the secondary sensing agent comprises a cleavable oligonucleotide linker.

34. The method of claim 33, wherein the secondary sensing agent is attached to a surface support via the linker.

35. The method of claim 34, wherein the surface support is located on a paper flow strip, a bead, or a microfluidic channel.

36. The method of any one of claims 34-35, further comprising, detaching the secondary sensing agent from the surface support by cleaving the cleavable oligonucleotide linker with the activated primary sensing agent.

37. The method of any one of claims 25-36, wherein the oligonucleotide linker further comprises a scrambled sequence.

38. The method of claim 37, wherein the scrambled sequence remains attached to the secondary sensing agent after linker cleavage by a unrelated enzyme in the biological sample.

39. The method of claim 38, wherein the secondary sensing agent comprising the scrambled sequence is filtered out of the sample and not detected.

40. The method of claim 24, wherein the filtering comprises hybridizing the recognition sequence with a complementary or partially complementary oligonucleotide attached to a surface.

41. The method of claim 9, wherein the detecting step is performed by a detector.

42. The method of claim 41, wherein the detector comprises a photonic or plasmonic waveguide.

43. The method of claim 42, wherein the waveguide is an optical ring resonator or a unbalanced Mach-Zehnder interferometer

44. The method of claim 9, wherein the second reaction results in a reaction product and the reaction product is captured by components bound to a detector, thereby resulting in a measurable signal.

45. The method of claim 9, wherein the detecting step comprises detecting changes in refractive index, electrical voltage, electrical current, optical absorbance, color, fluorescence, weight, melting temperature, or chemiluminescence.

46. The method of claim 43, wherein the detecting step comprises detecting changes in refractive index.

47. The method of claim 44, wherein the changes in refractive index are at the surface of a photonic or plasmonic waveguide.

48. The method of any one of claims 41-47, further comprising, contacting a binding agent or probe located on the detector with the reaction product.

49. The method of claim 48, wherein said probe or binding agent is an antibody, an antigen, or an aptamer.

50. The method of claim 49, wherein said probe or binding agent comprises an optically active component.

51. The method of claim 50, wherein said optically active component is a plasmonic nanoparticle, a gold nanoparticle, a quantum dot, or a fluorophore

52. The method of claim 9, wherein a component of said target biomolecule activates a cleaving component in the primary sensing agent.

53. The method of claim 9, wherein the secondary sensing agent is dried and is located within a high-surface area.

54. The method of claim 9, wherein the biological sample is placed in a reaction chamber comprising a high surface area and volume, and comprising a plurality of primary sensing agents.

55. The method of claim 54, wherein the plurality of primary sensing agents in the reaction chamber is activated by the target biomolecule.

56. The method of claim 9, the method further comprising, moving the sample through a microfluidic channel for sample detection.

57. The method of claim 9, the method further comprising, filtering the sample before sample detection.

58. The method of claim 9, further comprising a tertiary sensing agent bound by a second linker to a second surface, wherein said activated primary sensing agent catalyzes a third reaction that results in activation of said tertiary sensing agent comprising a third catalytic activity.

59. The method of claim 58, wherein the activated secondary sensing agent binds and cleaves the second linker and the activated tertiary sensing agent binds and cleaves the first linker, thereby cleaving a plurality of secondary and tertiary sensing agents from the first and second surfaces.

60. A system for detecting a target biomolecule in a biological sample, the system comprising:
a primary sensing agent activatable by the target biomolecule;
a secondary sensing agent, comprising a linker comprising a cleavage site cleavable by the activated primary sensing agent; and wherein the secondary sensing agent is activatable by the linker cleavage;
a detector, wherein the detector is capable of sensing the activated secondary sensing agent, thereby detecting the presence of said target biomolecule in said biological sample.

61. The system of claim 60, wherein the target biomolecule is a biological marker.

62. The system of claim 60, wherein the target biomolecule is selected from the group consisting of a dsDNA, a ssDNA, a RNA, a peptide, a protein, an antigen, and a pathogen.

63. The system of claim 60, wherein the target biomolecule comprises a RNA or a DNA.

64. The system of any one of claims 60-63, wherein the biological sample is selected from the group of blood, cord blood, saliva, mucus, tissue, nasal swab, and urine.

65. The system of any one of claims 60-64, wherein the primary sensing agent comprises a CRISPR enzyme.

66. The system of claim 65, wherein the CRISPR enzyme comprises a Cas12 complex or a Cas13 complex.

67. The system of claim 66, wherein the CRISPR enzyme comprises a Cas12a complex.

68. The system of claim 66, wherein the CRISPR enzyme comprises a Cas13 selected from the group consisting of a LwaCas13a, a CcaCas13b. a LbaCas13a, and a PsmCas13b.

69. The system of any one of claims 60-68, wherein the secondary sensing agent is an enzyme selected from the group consisting of a restriction enzyme, a hydrolase, a peroxidase, a lyase, a ligase, a glutathione S-transferase, and a SpyTag/SpyCatcher.

70. The system of claim 60, wherein the secondary sensing agent has nuclease activity.

71. The system of claim 60, wherein the secondary sensing agent comprises a CRISPR enzyme.

72. The system of claim 71, wherein the CRISPR enzyme comprises a Cas12 complex or a Cas13 complex.

73. The system of claim 72, wherein the CRISPR enzyme comprises a Cas12a complex.

74. The system of claim 72, wherein the CRISPR enzyme comprises a Cas13 selected from the group consisting of a LwaCas13a, a CcaCas13b. a LbaCas13a, and a PsmCas13b.

75. The system any one of claims 60-74, wherein the secondary sensing agent is an binding protein selected from the group consisting of an avidin, a maltose-binding protein, and a chitin-binding protein.

76. The system any one of claims 60-75, wherein activation of said secondary sensing agent comprises cleaving a first linker binding said secondary sensing agent to a first surface.

77. The system of any one of claims 60-75, wherein activation of said secondary sensing agent comprises cleaving an inactivating linker on said secondary sensing agent.

78. The system any one of claims 76-77, wherein the linker comprises ssDNA, dsDNA, RNA, or a combination thereof.

79. The system of claim 76, wherein the linker comprises an oligonucleotide or a modified oligonucleotide.

80. The system any one of claims 76-79, wherein the linker comprises a cleavage or recognition site for the primary sensing agent.

81. The system of claim 80, wherein the recognition or cleavage site comprises a TTA sequence or a TTA sequence repeat.

82. The system of claim 80, wherein the recognition or cleavage site comprises a sequence selected from the group consisting of AU, UC, AC, and GA.

83. The system of claim 76, wherein the linker is a peptide linker.

84. The system of claim 60, wherein the secondary sensing agent comprises a cleavable oligonucleotide linker.

85. The system of claim 84, wherein the secondary sensing agent is attached to a surface support via the linker.

86. The system of claim 85, wherein the surface support is located on a paper flow strip, a bead, or a microfluidic channel.

87. The system of any one of claims 85-86, further comprising, detaching the secondary sensing agent from the surface support by cleaving the cleavable oligonucleotide linker with the activated primary sensing agent.

88. The system of any one of claims 76-87, wherein the oligonucleotide linker further comprises a scrambled sequence.

89. The system of claim 87, wherein the scrambled sequence remains attached to the secondary sensing agent after linker cleavage by a unrelated enzyme in the biological sample.

90. The system of claim 87, wherein the secondary sensing agent comprising the scrambled sequence is filtered out of the sample and not detected.

91. The system of claim 90, wherein the filtering comprises hybridizing the recognition sequence with a complementary or partially complementary oligonucleotide attached to a surface.

92. The system of claim 60, wherein the detector comprises a photonic or plasmonic waveguide.

93. The system of claim 92, wherein the waveguide is an optical ring resonator or a unbalanced Mach-Zehnder interferometer.

94. The system of claim 60, wherein the detector measures changes in refractive index, electrical voltage, electrical current, optical absorbance, color, fluorescence, weight, melting temperature, or chemiluminescence.

95. The system of claim 94, wherein the detector measures changes in refractive index.

96. The system of claim 95, wherein the changes in refractive index are at the surface of a photonic or plasmonic waveguide.

97. The system of any one of claims 60-96, further comprising a binding agent or probe located on the detector.

98. The system of claim 97, wherein said probe or binding agent is an antibody, an antigen, or an aptamer.

99. The system of claim 98, wherein said probe or binding agent comprises an optically active component.

100. The system of claim 99, wherein said optically active component is a plasmonic nanoparticle, a gold nanoparticle, a quantum dot, or a fluorophore.

101. The system of claim 60, wherein a component of said target biomolecule is capable of activating a cleaving component in the primary sensing agent.

102. The system of claim 60, wherein the secondary sensing agent is dried and is located within a high-surface area.

103. The system of claim 60, further comprising a reaction chamber comprising a high surface area and volume, and comprising a plurality of primary sensing agent for placing the biological sample.

104. The system of claim 60, wherein the plurality of primary sensing agents in the reaction chamber is activatable by the target biomolecule.

105. The system of claim 60, the system further comprising, a microfluidic channel for moving the sample for sample detection.

106. The system of claim 60, the system further comprising a filter, for filtering the sample before sample detection.

107. The system of claim 60, further comprising a tertiary sensing agent bound by a second linker to a second surface, wherein said activated primary sensing agent is capable of catalyzing a third reaction resulting in activation of said tertiary sensing agent comprising a third catalytic activity.

108. The system of claim 107, wherein the activated secondary sensing agent is capable of binding and cleaving the second linker and the activated tertiary sensing agent is capable of binding and cleaving the first linker, thereby resulting in cleavage of a plurality of secondary and tertiary sensing agents from the first and second surfaces.

109. A method for assaying a biological sample suspected of comprising a target biomolecule, comprising:
obtaining a biological sample in a cartridge, wherein the cartridge comprises a sensor photonic integrated subcircuit;
contacting the sample with a primary sensing agent, wherein the primary sensing agent is activated in the presence of said target biomolecule;
providing conditions for activating a secondary sensing agent in the presence of said activated primary sensing agent, wherein said activated primary sensing agent comprises a first catalytic activity that results in activation of said secondary sensing agent comprising a second catalytic activity;
providing conditions for detecting a signal agent, wherein said activated secondary sensing agent comprises a second catalytic activity for a reaction that results in detection of said signal agent;
positioning the cartridge relative to an interrogator photonic circuit such that the cartridge is optically coupled with the interrogator photonic circuit, wherein the interrogator photonic circuit comprises (i) a light source configured to generate light, (ii) a waveguide configured to carry the light, and (iii) a photodetector configured to detect said light after passing through said waveguides; and
determining, via the light, a characteristic of the biological sample in the cartridge thereby detecting a quantity of a signal generated by said signal agent, thereby assaying said biological sample for said target biomolecule.

110. The method of claim 109, wherein said characteristic of the biological sample is determined based on a change in resonance, interference, or absorption caused by the biological sample.
